(19) United States
(12) Patent Application Publication
(10) Pub. No.: US 2011/0092933 A1
(43) Pub. Date: Apr. 21, 2011

(54) Environmentally-Friendly Disposable Absorbent Article

(75) Inventors: Carlos Canales Espinosa de los Monteros, Puebla (MX); Andrés Zamudio Ahumada, Puebla (MX); Lucia Sánchez Fernández, Puebla (MX)

(73) Assignee: GRUPO P.I MABE, S.A. DE C.V., Colonia La Loma, Puebla (MX)

(21) Appl. No.: 12/991,857

(22) PCT Filed: May 8, 2009
(86) PCT No.: PCT/IB2009/005526

§ 371 Date: Nov. 9, 2010

(30) Foreign Application Priority Data
May 9, 2008 (MX) MX/A/2008/006155

(51) Int. Cl. A61L 15/60; A61F 13/53; A61L 15/28; A61L 15/46

(52) U.S. Cl. 604/359; 604/372; 604/374; 604/377

(57) ABSTRACT

The invention relates to an environmentally-friendly disposable absorbent article. At least half of the constituent materials used in the disposable absorbent article contain biodegradable compounds and/or compounds of natural origin.

### BACKGROUND

A disposable absorbent article is a product that is used to absorb and contain body exudates and which is discarded after use, like disposable baby diapers, disposable underwear such as training pants and swimming pants, and all kinds of adult incontinence, sanitary napkins and panty liners, among others.

The disposable absorbent articles are used to protect the wearer's clothing, preventing it from getting dirty with body waste. The use of such articles has become indispensable in human daily life. However, most of the materials used to manufacture these disposable items are not sustainable neither biodegradable, it takes hundreds of years return them to nature.

A sustainable material is based on renewable resources, such that it helps to satisfy the needs of present generations without committing the resources of future generations, while a biodegradable material is that which is made from naturally occurring compounds, so that after certain period of time it breaks down and transforms in less complex compounds, until it reaches its most basic components.

The disposable absorbent articles such as disposable diapers, are generally made of a permeable top sheet, an impermeable back sheet (which may consist of two or more films laminated together), an absorbent core placed between the top and back sheets containing fibres of absorbent material and particles of superabsorbent material, external barrier cuffs, a fastening system, means of elastification in waist and legs areas, and union means, the article may also contain one or more transfer layers, one or more wrap layers and one or more elastic zones, in addition to perfume and/or some kind of formula for skin care and for control bad odour. Most of these components are made from polymers derived from petroleum, such as polyethylene, polypropylene, polyester, etc. For purposes of the present invention, a component of the article that contains such polymers will be referred as polymeric components of the article. Thus, the polymeric components of a disposable absorbent article, such as a disposable diaper are: top sheet, back sheet, fastening system, barrier cuffs, transfer layers, wrap layers, elastic leg and waist bands; that is to say, approximately 80% of the components of a disposable absorbent article, such as a disposable diaper, are polymeric components, and are neither sustainable nor biodegradable.

In addition, the superabsorbent material commonly used in the absorbent core of such absorbent articles, is generally a sodium or potassium polyacrylate, material not sustainable and not biodegradable.

All the materials can be joined using ultrasonic bonds, by means of heat and pressure, or using hot melt adhesives. In the latter case, the adhesives are neither sustainable nor biodegradable.

Thus, a disposable absorbent article, such as a disposable diaper, contains between 8 and 15 components, in addition to the bag in which the articles are packed. Based on eight components, namely, top sheet, back sheet, barrier cuffs, absorbent core, superabsorbent material, fastening system and union means, currently only one of them, the absorbent material, is sustainable and biodegradable, i.e. ⅛ or 12.5%.

The present invention relates to a disposable absorbent article in which at least 50% of the components are sustainable or biodegradable materials that is to say, a disposable absorbent article environmentally friendly, in addition this article satisfies consumer expectations in terms of absorbency, comfort and cost. An important aspect of the present invention is that the materials used are suitable for processing them at high production rates used on a daily basis for the manufacture of such articles.

There are many developments and proposals for use of biodegradable materials in absorbent articles. Some of the most important are described in patents and publications U.S. Pat. No. 4,964,857, U.S. Pat. No. 5,185,009, WO 07/106,929, WO 07/106,929, U.S. Pat. No. 5,437,918, US2005/0112363, U.S. Pat. No. 6,506,873, JP 07008520, U.S. Pat. No. 5,417,679, WO 01/39 807, U.S. Pat. No. 5,295,985. U.S. Pat. No. 5,939,467, WO 02/028444, WO 05/094910, WO 99/33420 and the application MX 9,911,256

However the large number of developments in this field, it is difficult to find in the market environmentally friendly disposable absorbent articles. This is because the available materials:
Are not suitable for use in the manufacturing of such items (do not have the needed strength or the needed weight)
Do not have the right characteristics in terms of softness, flexibility and security for the user.
Are expensive compared with commonly used materials.

For example, U.S. Pat. No. 4,964,857 of Charles Osborne, entitled “Biodegradable Disposable Diaper”; refers to a diaper that has a renewable top sheet and a back sheet of paper coated with wax, which helps prevent fluid to flow through itself. However, it is clear that a product of this nature is not sure and not comfortable for the user, disposable absorbent articles with these characteristics have no place in the market. Another development in this regard is U.S. Pat. No. 5,185,009 of Elmo Sitman, which describes a biodegradable diaper with a top sheet, a back sheet, an absorbent core and adhesives; adhesive tapes are made of cellulose and covered with a biodegradable adhesive; the top sheet can be biodegradable rayon or polypropylene, the back sheet can be a biodegradable polyethylene, the bonding adhesive is latex rubber, the core layer is absorbent wood pulp or a copolymer of poly-acrylic-nitrite and retaining straps are coated paper tape. A diaper of this type not has been marketed as such, since the described materials are not adequate to provide the consumer an article with the features he expects in terms of absorbency, comfort and safety. The international publication WO 06/065110, of Estela Concepcion Gonzalez Flores, refers to a disposable absorbent article which uses a natural waterproofing agent applied to a film, such as paper or nonwoven fabric, the article also uses a re-useable waterproof covering. The international publication WO 07/106,929, of Tristano PTY LTD, describes a disposable absorbent article fabricated with biodegradable materials made from one or more biodegradable polymers such as poly-Capra-lactone and mixtures of starch and PLA, the article contains others seeds embedded in the absorbent core, so that after used, it is buried and the seeds germinate. All remaining components of the article, such as elastic and adhesive fastening system may also be biodegradable.

The present invention provides a disposable absorbent article, such as a disposable diaper, a training pants, a pad or any similar article in which at least half of its components contain sustainable or biodegradable materials; the article is comfortable, safe and affordable, plus it can be manufactured on production lines of high speed. So in the absorbent article of the present invention, all polymeric components are replaced by polymeric materials that can be chosen from:
Films or materials containing polylactic acid (PLA) or polyhydroxy-alcanoate (PHA)
Films or materials containing natural fibres such as cotton or rayon.
Films or polymeric materials containing a pro-degrading agent.

Moreover, the superabsorbent material, which is commonly sodium or potassium polyacrylate, is replaced by a mixture containing at least 20% of absorbent polymer from corn starch particles in combination with polyacrylate.

Thus, is available to the consumer, a environmentally friendly product that has the right features in absorption, comfort, safety and smoothness, at an affordable cost and also it can be manufactured on production lines at high speed.

In what refers to the use of natural fibres such as cotton or rayon to produce films that can be used in this articles, it is noteworthy that there are some developments in nonwoven fabric containing cotton fibres, however most refers to fabrics suitable for cleaning, due to their great absorbent capacity, such as described in WO 91/08333 which protect a method for manufacturing hydrophilic nonwoven fabric containing natural fibres, specifically unbleached cotton, a fabric of this type is not suitable for be used as top layer of a disposable absorbent article due to its high degree of hydrophilicity. In disposable absorbent articles it is desirable the use of a permeable top layer to allow the passage of fluid but it should not remain wet, meaning that the liquid passes through and is distributed, absorbed and retained by inner layers of the article, keeping moisture away from the user's skin; a nonwoven fabric as described in WO 91/08333, absorbs and retains moisture, so it is not appropriate for its use in absorbent articles.

Other developments of nonwoven fabric containing natural fibres are used as absorbent material, either in disposable sanitary articles, or in other products, such as food packaging, etc., Fabrics suitable for this use are protected in U.S. Pat. No. 5,958,186 SCA Hygiene Products and U.S. Patent Application 2003/007026 Jens O. Brøchner.

On the other hand, non-woven fabrics containing natural fibres can be found as filling for mattresses, duvets, pillows, etc. this type of fabrics are described in U.S. Patent Application 2003/0021978 by Anthony Wolf.

None of the films described is suitable for use as a top sheet of disposable sanitary articles. We propose the use in a disposable absorbent article of a nonwoven fabric containing at least 15% of a natural fibres such as cotton, mixed with other synthetic fibres, so that the fabric is permeable but does not retain moisture, has adequate resistance to withstand the stresses to which it is subjected during the manufacturing process and during use, plus it is smooth.

### SUMMARY

The main object of the present invention is to provide the consumer with an environmentally friendly disposable absorbent article, in which at least half of the materials used to manufacture them, contain natural or biodegradable compounds, therefore, raises several additional objectives:
The use as top sheet of the article, a nonwoven fabric containing cotton.
The use as top sheet of the article, a nonwoven fabric containing polylactic acid (PLA) or poly-hydroxy-alcanoates (PHA).
The use as top sheet of the article, a nonwoven fabric containing a pro-degrading agent.
The use as back sheet of the article, a waterproof film containing polylactic acid (PLA) or poly-hydroxy-alcanoates (PHA).
The use as back sheet of the article, a polyethylene containing a pro-degrading
The use as back sheet of the article, a laminated layer of a film containing PLA, PHA or a pro-degrading agent and:
   A nonwoven fabric containing cotton.
   A nonwoven fabric containing PLA or PHA
   A nonwoven fabric containing a pro-degrading agent.
The use of superabsorbent material containing at least 20% of natural and/or biodegradable particles.
The use of a fastening system containing PLA or PHA.
The use of a fastening system containing a pro-degrading agent.
The use of films containing PLA or PHA or a pro-degrading agent for barrier cuffs, transfer layers and wrap layers.
The use of Adhesives containing PHA or PLA.
The use of elastic or elastomeric materials containing PLA, PHA or pro-degrading agent.

### BRIEF DESCRIPTION OF THE DRAWINGS

The invention will be described with reference to the accompanying drawing, which illustrates a presently preferred embodiment of the invention.

FIG. 1 shows a disposable absorbent article.

### DETAILED DESCRIPTION

The term disposable absorbent article refers to articles which absorb and retain body exudates and are discarded after use. It is protected by the present invention a disposable absorbent article which is friendly to nature, such that at least 50% of the total materials used to manufacture them, contain natural and/or biodegradable compounds.

This description will be based on a disposable baby diaper, understanding that can be applied to other disposable absorbent articles such as disposable diapers for incontinent adults, children's training pants, panty-liners, sanitary napkins or any other disposable absorbent article.

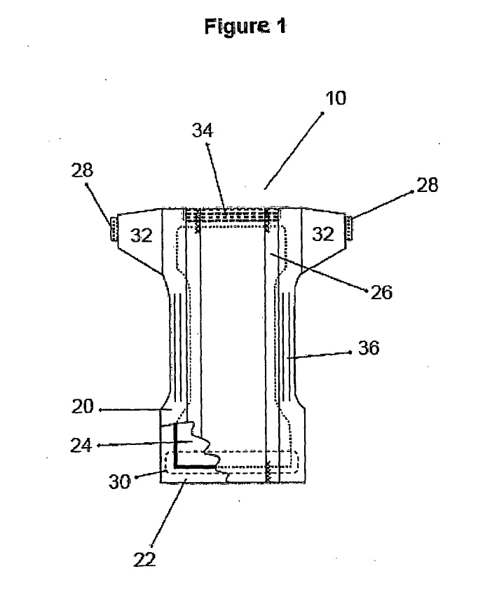

As seen in FIG. 1, the top sheet (20) of article (10) of the invention is preferably a nonwoven fabric containing at least about 50% of PLA or PHA, and the rest of a polyolefin, such as polyethylene or polypropylene; as mentioned in the background, the nonwoven containing PLA or PHA is currently available in the market, however, this kind of film is not suitable for use as top sheet of a disposable absorbent article, mainly because the PLA and PHA do not provide the necessary smoothness for this type of articles, besides, to be affordable, the weights of the films used as top sheet are handled in 10 to 15 g/m². Available nonwoven fabrics having this weight and made of degradable polymers such as PLA or PHA, do not have the strength and elongation required to work well in the processes of producing such articles. In addition, disposable absorbent articles, during use, are in direct contact with the skin all day and night, the films made from polymers such as PLA and PHA do not have the smoothness required for no hurt the skin.

The nonwoven fabric used as top sheet (20) in article (10) of this invention is made from bicomponent fibres of sheath-core type, the core contains about 50 to about 100% of PLA or PHA, preferably from about 80 to about 100% of PLA or PHA while the sheath contains about 50 to about 100% polypropylene, preferably from about 80 to about 100% polypropylene, the remaining contents of the sheath may be PLA or PHA. This nonwoven fabric used as top sheet (20) in Article (10) invention contains at least 50% of sustainable materials.

For the purposes of the present invention, it can also be used as top sheet, a nonwoven fabric containing natural fibres, such as cotton. In this regard, there are several developments, however, no such fabrics are used in disposable absorbent articles, since cotton is a hydrophilic fibre, so it absorbs and retains liquid, undesirable feature in the top sheet of a disposable absorbent article, therefore nonwoven fabrics containing cotton are used as cleaning cloths, for food packaging, as absorbent pads or even as a filling for mattresses. On the other hand, for the manufacture of absorbent disposable articles is required that the film used as top sheet and/or back sheet, has certain characteristics of machine direction and cross direction strength; a nonwoven fabric containing cotton, has less strength that a nonwoven fabric without it, a greater amount of cotton fibres in a non woven fabric, less strength. Thus, for the purposes of this invention, can be used as top sheet a nonwoven fabric containing between about 15 and 50% cotton fibre which are natural and biodegradable fibres blended with polyester or polypropylene.

Can also be used as top sheet of the article of this invention, a nonwoven fabric made from polyolefins such as polypropylene or polyester which has added a pro-degrading agent, preferably the pro-degrading agent contains saponified fatty acids and calcium, so the film can be degraded in the presence or absence of oxygen, i.e. to compost or garbage dumps.

The back sheet (22) of article (10) of the present invention should be soft, flexible and waterproof, generally, is used low density polyethylene. For the purposes of article (10) of the invention, can be used as back sheet a low density polyethylene film containing a pro-degrading agent, preferably containing saponified fatty acids and calcium, so the film can be degraded in the presence or the absence of oxygen. It can also be used as backsheet or the article (10), a waterproof film made from PLA and/or PHA.

It is desirable that the back sheet (22) of article (10) has the appearance and softness of a fabric, to achieve this, is used a laminate of two films: polyethylene as described in the preceding paragraph, and nonwoven fabric as described for the top sheet (20).

The absorbent core (24) of article (10) of the invention, is constituted of fibres of absorbent material and particles of superabsorbent material; the absorbent material fibres are cellulosic fibres and the superabsorbent material particles are composed of a mixture of particles of sodium or potassium polyacrylate and at least 20% of absorbing particles derived from corn starch, this natural absorbent particles help the rapid dispersion of fluid in the core making it more efficient. Thus, in the absorbent core (24) of article (10) of the present invention, at least 20% of the particles of superabsorbent material are natural and biodegradable.

Another element of the disposable absorbent article (10) of the invention are the external barriers cuffs (26), these are made up of some of the films described above for the top sheet (20) with the proper treatment of hydrophobicity required for this element of the article.

The fastening system of disposable diapers is usually formed by a pair of fastening tapes (28), and a front band or tape (30); fastening tapes (28) are placed on the back of the diaper waist zone, while the front band or tape is placed on the outer layer on the frontal waist zone of the article. It can be used a mechanical (Hook & Loop) or adhesive system, in either case, both down straps or tapes as the front band, are usually made of no sustainable or biodegradable materials, usually polyethylene or polypropylene, so, for the fastening system of the present invention are used preferably a couple of bands (28) (mechanical or adhesive) and a front band or tape (30), which are made from degradable polymers such as PHA and PLA, or from polyethylene or polypropylene containing a pro-degrading agent; preferably the pro-degrading agent contains saponified fatty acids and calcium.

An absorbent article may contain one or more transfer layers and one or more wrap layers, for the absorbent article of the present invention, as transfer or wrap layers are used nonwoven films such as those described for the upper layer.

Moreover, the absorbent article has elastic means (36 and 34) at least in the part that is in contact with the wearer's legs and in the part of the waist, for this purpose, usually, are used lycra, polyester elastic bands, films of polypropylene or polyurethane, which are bonded, in a state of tension, between two layers of the article, generally between the top and backsheet layers; for the purposes of the present invention, as elastic means in legs or waist zones, can be used latex or natural rubber; can also be used elastic materials made from polyolefin plus a pro degrading agent.

Article (10) may also have other stretch zones located in different parts of the article, for example in the sides zones, in the ears (32) or as an integral part of the fastening bands (28). For the article (10) of the invention, these elastic areas could be formed by a laminate consisting of a couple of films containing, between them, an elastomeric material, which can be bonded to the films with adhesive, ultrasonic, thermal bond or extruded directly onto one of the films. For the purposes of the invention, the elastomeric material contains a pro-degrading agent and the films are films like those described for topsheet or backsheet of the article (10) of the invention.

To bond all the components of the article (10), can be used thermal, ultrasonic or adhesive bonds. The components of the absorbent disposable articles commonly found on the market, are bonded by means of adhesives, specifically hot melt and fast dry adhesives. For the purposes of the invention can be used adhesives made from degradable polymers such as PLA or PHA.

As additional elements, absorbent articles often contain perfume and/or a formula to control odour and/or a formula to protect the user's skin; for the article of the invention, is used an essential oil blend containing chamomile, lavender, geranium, ylang ylang and eucalyptus to protect user's skin and cupric chlorophyll as an agent for odour control, all naturally occurring compounds.

### EXAMPLES

### Example 1

Top Sheet: nonwoven fabric of bi-component fibres (PLA core covered with polypropylene), with a weight of 13 g/m2.
Back Sheet: laminated film formed of 15 g/m2 biodegradable polyethylene (polyethylene+pro degrading agent containing saponified fatty acids and calcium) and nonwoven fabric of 13 g/m2 composed of polypropylene/cotton fabric in a 70/30 ratio.
Absorbent Material: cellulose fibres.
Superabsorbent Material: a mixture of polyacrylate particles and particles of corn starch in a ratio 80/20.
External Barrier cuffs: nonwoven fabric of 13 g/m2 composed of bicomponent fibres (PLA core covered with polypropylene).
Emollient: Formula based on vegetable oils, mainly avocado oil and sweet almond oil.
Fastening System: mechanical Hook & Loop fastening system, made of polyolefins.
Leg and Waist elastic: lycra
Union means: hot melt adhesives.

Whereas the back sheet is formed by two films joined together and that the fastening system is formed by fastening bands and frontal band, the diaper is formed by a total of 11 elements, seven of which contain sustainable or biodegradable compounds, i.e. 63.6% of the components of the disposable diaper of Example 1 contain sustainable or biodegradable compounds.

### Example 2

Top Sheet: non-woven fabric of 15 g/m2 composed of 20% Cotton fibres and 80% polypropylene fibres.
Back Sheet; polyethylene of 15 g/m2 plus a pro-degrading agent containing saponified fatty acids and calcium, laminated with a non-woven fabric of 13 g/m2 composed of polypropylene/cotton fibres in a 70/30 ratio.
Absorbent Material: cellulose fibres.
Superabsorbent Material: a mixture of polyacrylate particles and particles of corn starch in a 80/20 ratio.
External Barrier Cuffs: nonwoven fabric of 13 g/m2 composed of bicomponent fibres (PLA core covered with polypropylene).
Emollient: Formula based on chamomile oil, lavender oil, geranium oil, ylang ylang oil and eucalyptus oil plus cupric chlorophyll to control odours.
Fastening System: Hook & Loop mechanical system made of polyolefins plus a pro-degrading agent.
Legs and waist elastics: lycra.
Union means: Hot melt adhesives made from PLA.

Total components: 11. Sustainable or biodegradable materials: 9=81.8%

### Example 3

Top Sheet: non-woven fabric of 13 g/m2 composed of 20% Cotton and 80% polypropylene fibres.
Back Sheet: 13 g/m2 polyethylene containing a pro-degrading agent, laminated with a non-woven fabric of 13 g/m2 with 20% of cotton fibres.
Absorbent Material: cellulose fibres.
Superabsorbent Material: a mixture of polyacrylate particles and particles of corn starch in a ratio 80/20.
Transfer Layer: polypropylene nonwoven fabric plus a pro-degrading agent.
External Barrier Cuffs: nonwoven fabric of 13 g/m2 composed of bicomponent fibres (PLA core covered with polypropylene).
Emollient: Formula formed with essential oils.
Fastening System: Hook & Loop mechanical fastening system made of polyolefins plus a pro-degrading agent.
Union means: Hot melt adhesives

Total component: 12. Sustainable or biodegradable materials: 11=91.6%

### Example 4

Top Sheet: nonwoven fabric of 13 g/m2 composed of 20% Cotton and 80% polypropylene fibres.
Back Sheet: 13 g/m2 laminated film composed of polyethylene containing a pro-degrading agent and non-woven fabric of 13 g/m2 with 20% of cotton fibres.
Absorbent Material: cellulose fibres.
Superabsorbent Material: a mixture of polyacrylate particles and particles of corn starch in a ratio 80/20.
Wrap Layer: paper of 15 g/m2
Transfer Layer: Polypropylene non-woven fabric of 18 g/m2 containing a pro-degrading agent.
External Barrier Cuffs: nonwoven fabric of 13 g/m2 composed of bicomponent fibres (PLA core covered with polypropylene).
Emollient: Formula containing essential oils and chlorophyll.
Fastening System: Hook & Loop mechanical fastening system made of polyolefins containing a pro-degrading agent.
Stretch Zones: biodegradable elastomer (polyolefin plus a por-degradent agent) laminated between two layers of nonwoven fabric of bi-component fibres (PLA core covered with polypropylene) with a weight of 13 g/m2.
Legs and waist elastic: lycra or polyurethane foam plus a pro-degrading agent.
Union means: Hot melt adhesives based on PLA and/or PHA polymers.

Total components: 14 Sustainable or biodegradable materials: 14=100%

1. A disposable absorbent article comprising:
a permeable top sheet;
an impermeable back sheet;
an absorbent core placed between the top sheet and the back sheet, the absorbent core containing absorbent material fibers and particles of superabsorbent material;
external barriers cuffs;
waist and leg elastic portions;
a fastening system; and
a bonding system,
wherein at least 50% of the disposable article comprises sustainable or biodegradable compounds.

2. The disposable absorbent article according to claim 1, wherein the permeable top sheet is a nonwoven fabric with a weight between 10 and 15 g/m², formed from bicomponent fibers of the core and sheath type, with the core containing 80 to 100% of polylactic acid and the sheath containing 80 to 100% of polypropylene.

3. The disposable absorbent article according to claim 1, wherein the permeable top sheet is a nonwoven fabric with a weight between 10 and 15 g/m², formed from bicomponent fibers of the core and sheath type, with the core containing 80 to 100% of poly-hydroxy-alcanoate lactic acid and the sheath containing 80 to 100% of polypropylene.

4. The disposable absorbent article according to claim 1, wherein the permeable top sheet is a nonwoven fabric with a weight between 10 and 15 g/m², containing at least about 20% of cotton fibers.

5. The disposable absorbent article according to claim 1, wherein the impermeable back sheet is a film of low density polyethylene with a weight of about 10-25 g/m², containing a pro-degrading agent which contains saponified fatty acids and calcium.

6. The disposable absorbent article according to claim 1, wherein the impermeable back sheet is a film made from polylactic acid.

7. The disposable absorbent article according to claim 1, wherein the impermeable back sheet is a film formed from poly-hydroxy-alcanoates.

8. The disposable absorbent article according to claim 1, wherein the impermeable back sheet is laminated with a nonwoven fabric with a weight between about 10 and 15 g/m² and containing bicomponent core and sheath fibers, with the core containing about 80 to about 100% polylactic acid and the sheath containing about 80 to 100% polypropylene.

9. The disposable absorbent article according to claim 1, wherein the impermeable back sheet is laminated to a nonwoven fabric with a weight between of about 10 and 15 g/m² and containing bicomponent core and sheath fibers, with the core containing about 80 to about 100% of poly-hydroxy-alcanoate and the sheath containing about 80 to 100% polypropylene.

10. The disposable absorbent article according to claim 1, wherein the impermeable back sheet is laminated to a nonwoven fabric with a weight between of about 10 and 15 g/m² containing at least about 20% of cotton fibers.

11. The disposable absorbent article according to claim 1, wherein the absorbent core is formed of cellulose fibers.

12. The disposable absorbent article according to claim 1, wherein the absorbent core is formed of cellulose fibers and a homogeneous mixture of:
a) particles of sodium or potassium polyacrylate; and
b) absorbent particles derived from corn starch.

13. The disposable absorbent article according to claim 12, wherein the mixture contains about 50 to about 95% of particles of sodium or potassium polyacrylate and about 5 to about 50% of absorbent particles derived from corn starch.

14. The disposable absorbent article according to claim 1, wherein the external barrier cuffs are formed from a film of nonwoven fabric with a weight between about 10 and about 15 g/m², formed of bicomponent core and sheath fibers, with the core containing about 80 to about 100% polylactic acid and the sheath containing about 80 to about 100% polypropylene.

15. The disposable absorbent article according to claim 1, wherein the external barrier cuffs are formed from a nonwoven fabric with a weight of about 10 to about 15 g/m², formed of bicomponent core and sheath fibers, with the core containing about 80 to about 100% poly-hydroxy-alcanoates and the sheath containing about 80 to about 100% polypropylene.

16. The disposable absorbent article according to claim 1, wherein the external barrier cuffs are formed from a nonwoven film with a weight of about 10 to about 15 g/m² containing at least 15% of cotton fibers.

17. The disposable absorbent article according to claim 1, wherein the leg and waist elastic portions of the article are latex or natural rubber.

18. The disposable absorbent article according to claim 1, wherein the leg and waist elastic portions of the article are made from polyolefins plus a pro-degrading agent.

19. The disposable absorbent article according to claim 1, wherein the fastening system of the article comprises a pair of fastening tapes placed on a rear waist portion of the article and a band placed outside of a front waist portion thereof, wherein at least one of the fastening tapes and the band is formed of polyolefins plus a pro-degrading agent containing saponified fatty acids and calcium.

20. The disposable absorbent article according to claim 1, wherein the fastening system comprises a pair of fastening tapes placed on a rear waist portion of the article and a band placed the outside of a front waist portion thereof, wherein at least one of the fastening tapes and the band contains polylactic acid or poly-hydroxy-alcanoates.

21. The disposable absorbent article according to claim 1, wherein the article comprises stretch zones comprising an elastomeric material placed between two films, wherein the elastomeric material is a polyolefin plus a pro-degrading agent containing saponified fatty acids and calcium, and the films are nonwoven films.

22. The disposable absorbent article according to claim 1, wherein the article comprises stretch zones comprising an elastomeric material placed between two films, wherein the elastomeric material is a polyolefin plus a pro-degrading agent containing saponified fatty acids and calcium, and the films are nonwoven fabrics of about 10-15 g/m², which are formed by bicomponent core and sheath fibers, with the core containing about 80 to about 100% of polylactic acid and the sheath containing about 80 to 100% of polypropylene.

23. The disposable absorbent article according to claim 1, wherein the article comprises stretch zones comprising an elastomeric material placed between two films, wherein the elastomeric material is a polyolefin plus a pro-degrading agent containing saponified fatty acids and calcium, and the films are nonwoven fabrics of about 10-15 g/m², which are formed by bicomponent core and sheath fibers, with the core containing about 80 to about 100% of poly-hydroxy-alcanoate and the sheath containing about 80 to 100% of polypropylene.

24. The disposable absorbent article according to claim 1, wherein the article comprises stretch zones comprising an elastomeric material placed between two films, the elastomeric material is a polyolefin plus a pro-degrading agent containing saponified fatty acids and calcium, and the films are nonwoven fabrics of about 10-15 g/m², containing at least about 20% cotton fibers.

25. The disposable absorbent article according to claim 1, wherein the article comprises a composition for skin care containing vegetable oils.

26. The disposable absorbent article according to claim 1, wherein the article comprises a composition for skin care containing a mixture of essential oils.

27. The disposable absorbent article according to claim 1, wherein the article comprises chlorophyll or chlorophyll derivatives in an amount effective for odor control.
